(19) United States
(12) Patent Application Publication
(10) Pub. No.: US 2014/0066871 A1
(43) Pub. Date: Mar. 6, 2014

(54) Anatomically Conforming Vaginal Insert With Cover

(71) Applicants: Kristin M. Shepard, Appleton, WI (US); MaryAnn Zunker, Oshkosh, WI (US)

(72) Inventors: Kristin M. Shepard, Appleton, WI (US); MaryAnn Zunker, Oshkosh, WI (US)

(21) Appl. No.: 13/601,085

(22) Filed: Aug. 31, 2012

(51) Int. Cl. A61F 13/15

(52) U.S. Cl. CPC A61F 13/15203
USPC 604/364

(57) ABSTRACT

An anatomically-conforming vaginal insert is formed from a composition, wherein the composition includes a material that is introduced into a vaginal canal as a viscous material at a first temperature and after insertion into the vaginal canal becomes a semi-solid at a second temperature greater than the first temperature, wherein the first temperature is about 20° C. and the material has increased solidity at the second temperature as compared to the first temperature, and wherein the composition is disposed within a over sheet. The vaginal insert can be formulated with a withdrawal device, and the withdrawal device can be attached to the cover sheet.

### BACKGROUND

The present disclosure relates to a composition, device, and a method of delivery for an anatomically conforming vaginal insert with a cover sheet.

There have been numerous devices developed to address issues of urinary incontinence and menstruation. Many of these devices are generally round, rectangular, or ovoid in shape. However, the vagina is a hollow fibro-muscular, non-cylindrical tube in which the right and left lateral walls form what resembles an “H” shape with the anterior and posterior walls collapsed upon each other. Additionally, the surface of the vagina is not smooth; there are small ridges on the inner surface of the vagina that extend laterally and upward from the columna rugarum (the long ridges on the anterior and posterior walls). Therefore, while the above devices are successful, it is believed that they could be improved to better take into consideration the shape and internal structures of the vagina. The present disclosure addresses that need.

### SUMMARY

In accordance with the present disclosure, therefore, an anatomically conforming vaginal insert is provided. The insert is desirably flushable, discardable, or biodegradable and is also biocompatible and suitable for placement in the human body. The insert is a composition that, prior to introduction into the vagina, has the physical properties of a liquid, semi-soft liquid, gel, paste, foam, or viscous material so that it can be easily and effectively delivered into the vaginal canal. After the material has been introduced into the vaginal canal, the composition will expand into a portion of the rugal folds and make intimate contact with a portion of the rugae where the consistency of the material will increase in solidity, forming a semi-solid or solid structure to substantially fill the entire “H” shaped space of The vagina within a portion of the vaginal canal. The intimate contact with the rugae ensures that the device will be securely held in place and additionally will not allow the passage of bodily fluids from the uterus or the urethra. Upon solidifying, the composition defines an insert that, in one aspect, is suitable for use as a tampon and, in another aspect, is suitable for use as a continence restoration device.

The composition can be delivered to the vagina by any suitable method including an aerosol, squeeze tube, pump or other applicators (known in the art). Additionally a suppository, capsule, effervescent or foam producing tablet could also be used to deliver the material into the vaginal canal.

As noted above, in one aspect, the insert can be used as a continence restoring device, to provide physical support to the bladder neck region by restoring it to normal anatomical position. Because the device substantially fills the entire “H” shaped space of the vagina within a significant portion of the vaginal canal, the device will also act as a backdrop to support the weakened musculature around the bladder neck and urethra to prevent or substantially reduce leakage. In addition, the insert will close off the urethra when increased abdominal pressure occurs, for example, upon coughing, laughing, exercising, lifting, or sneezing but does not interfere with the voluntary release of urine (micturation).

In this aspect, the composition is non-absorbent and desirably hydrophobic. The composition includes a material selected from polyurethane, silicone, temperature sensitive polymeric materials, ion sensitive polymeric materials, humidity activated materials having a shape memory.

In another aspect, the insert is contemplated as a catamenial device. In this aspect, the insert attracts and/or holds menstrual fluids. The composition forming the insert can be hydrophilic so long as the insert can attract and/or hold the menstrual fluid within the interstitial spaces of the material and prevent leaks.

In this aspect, the composition includes open-cell polyurethane foams, a multi-component reverse thermo-sensitive polymeric system such as that shown and described in PCT publication WO 03/017972, the contents of which are incorporated by reference, a fibrous absorbent structure resembling an open cell polymeric foam such as that shown and described in U.S. Pat. No. 6,261,679 the contents of which are incorporated herein by reference.

In one aspect of the present disclosure, an anatomically-conforming vaginal insert is formed from a composition, wherein the composition includes a material that is introduced into a vaginal canal as a viscous material at a first temperature and after insertion into the vaginal canal becomes a semi-solid at a second temperature greater than the first temperature, wherein the first temperature is about 20° C. and the material has increased solidity at the second temperature as compared to the first temperature, and wherein the composition is disposed within a cover sheet.

In another aspect of the present disclosure, an anatomically-conforming vaginal insert is formed from a composition that is introduced into a vaginal canal as a liquid, semi-soft liquid, gel, paste, foam or viscous composition at a temperature of about 16° to about 27° C., and after insertion into the vaginal canal becomes a solid or semi-solid material at a temperature of about 35° to about 39° C., wherein the composition is disposed within a cover sheet, and wherein the vaginal insert is formulated with a withdrawal device.

### DRAWINGS

The foregoing and other features and aspects of the present disclosure and the manner of attaining them will become more apparent, and the disclosure itself will be better understood by reference to the following description, appended claims and accompanying drawings, where:

FIG. 1 is a mid-sagittal section of a human torso showing one aspect of the material of the present disclosure deposited in the vaginal canal and acting as an incontinence device. The material fills the entire vaginal cavity and numerous rugae allowing restoration to nearly normal anatomical position furthermore, by cooperating with the symphysis pubis to allow the urethral tube to be compressed upon itself and alleviate urinary incontinence during episodes of increased intra-abdominal pressure.

FIG. 2 is a sectional view taken along line 2-2 of FIG. 1 with the material of the present disclosure not present within the vaginal cavity.

FIG. 3 is the same sectional view of FIG. 2, except that the material of the present disclosure is present within a portion of the vaginal cavity.

Repeat use of reference character in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

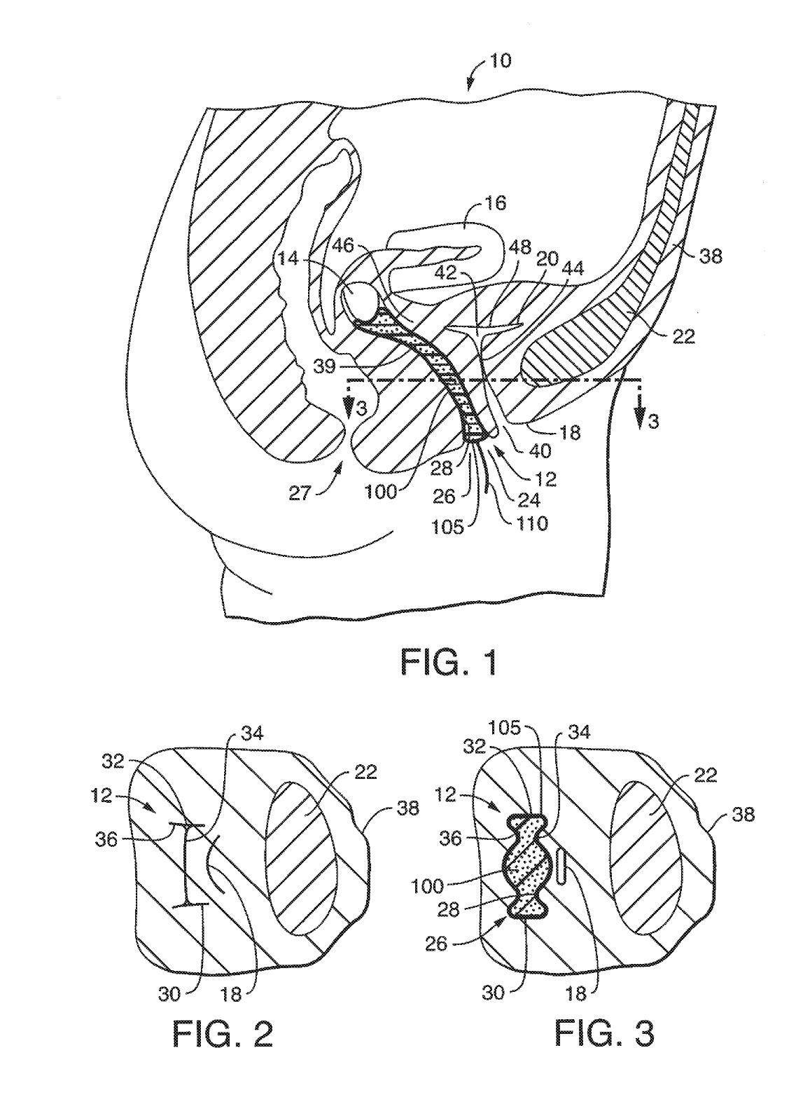

Turning now to FIG. 1, a human torso 10 of a female is shown with a vagina 12, a cervix 14, a uterus 16, a urethra 18, a bladder 20 and a symphysis pubis 22. The vagina 12 has an introital opening 24 that exits the human torso 10 that contains a vaginal anal 26 that extends from the introital opening 24 to the cervix 14. The vaginal canal 26 had a length that ranges from between about 4 inches to about 6 inches (about 102 millimeters (mm), to about 153 mm) in most women. The cervix 14 is the entrance to the womb and is located between the upper aspect of the vaginal canal 26 and the uterus 16. The rectum 27 is located posterior to the vagina 12. The vaginal canal 26 has an inner periphery 28.

As best seen in FIGS. 2 and 3, the inner periphery 28 is made up of a right lateral wall 30, a left lateral wall 32, an anterior wall 34, and a posterior wall 36. The four walls 30, 32, 34, and 36 generally define an H-shape and encompass the entire 360 degrees of the inner periphery 28. The inner periphery consists of rugal folds 39. The anterior wall 34 is located closest to the urethra 18 and the urethra 18 is located between the symphysis pubis 22 and the vagina 12.

The vaginal canal 26 can be divided into three approximately equal sections, each representing about one-third of the overall length. Each section is approximately 2 inches (approximately 51 mm) in length. The middle third of the vaginal canal 26 is typically the most important section for alleviating female urinary incontinence because of its proximity to the urethra 18. However, in the disclosure, the material fills the entire length of the vagina and penetrates into a portion of the rugal folds along its length making positioning in the middle third unimportant. The middle third of the vaginal canal 26 is also horizontally offset from the symphysis pubis 22, which is a bony prominence situated adjacent to a front portion 38 of the human torso 10. Cooperation between a urinary incontinence device positioned along the length of the vagina 12 and the symphysis pubis 22 further allows the urethra 18 to be compressed upon itself to alleviate involuntary urine flow from the bladder.

The urethra 18, also referred to as the urethral tube, is a hollow, tubular structure that extends from a first opening (urethral meatus) 40 that exits the human body 10 to a second opening 42 situated at the lower surface of the bladder 20. The urethra 18 has a length of about 1.5 inches (about 38 mm) in most women. The urethra functions to discharge urine, which is temporarily stored in the bladder 20, from the human body. The urethra 18 has a plurality of urethral sphincter muscles 44 located along the length of its inner periphery. The urethral sphincter muscles 44 are situated below the opening 42 and are ring like muscles that normally maintain constriction of the urethra 18 to prevent the passage of urine. The relaxation of the urethral sphincter muscles 44 by normal physiological functioning will permit urine to be voluntarily expelled from the body.

Again, referring to FIG. 1, the human torso 10 further includes musculature and body tissue located in the urethrovaginal myofascial area 46 that is situated between the vagina 12 and the symphysis pubis 22. The bladder 20 lies posterior to the symphysis pubis 22 and is separated from the rectum 27 by the vagina 12 and the uterus 16. The ureters (not shown) that transport urine from the kidneys to the bladder 20, pass from the pelvis to the posterior aspect of the urinary bladder 20. The fundus vesicae 48, into which both of the ureters terminate, is located adjacent to the anterior wall 34 of the vagina 12.

As shown in FIG. 1, according to one aspect of the present disclosure, the composition of the present disclosure is shown deposited within the vagina 12 and in the form of a disposable device 100 positioned in the vaginal canal 26. The disposable device 100 can be configured to be a urinary incontinence device or a catamenial tampon. in the aspect of a urinary incontinence device, the disposable device 100 is designed to bridge across and along the length of the vagina 12 to support the musculature and body tissue located in the urethra-vaginal myofascial area 46. it will be understood from the following description that the disposable device 100 will substantially fill the entire lateral 360 degree originally H-shaped space of the vagina 12 along the entire length of the vaginal canal 26.

FIG. 3 shows the disposable device 100, by virtue of its placement and composition, acting as a urinary incontinence device. It will be noted that the disposable device 100 substantially fills the entire space of the vagina 12 providing a supportive backdrop for the urethral 18. The urethrel 18 can now be sufficiently compressed during increased intra-abdominal pressure periods thereby intercepting the flow of urine and also to provide support to the urinary sphincter muscle 44 so that it can function properly. By permitting the urethral 18 to be compressed upon itself between the disposable device 100 and the symphysis pubis 22, the involuntary flow of urine from the bladder is limited.

As noted above, the composition of the present disclosure can be chosen to provide a urinary incontinence device or insert or to provide a catamenial tampon or insert. The composition desirably is a to surface tension material that will allow uniform expansion under relatively low pressure. As a result, the composition will expand then solidify when present in the vaginal canal 26 and will conform to the shape of the vagina 12, penetrating a portion of the folds of the mucous membrane (ruga) of the walls 30, 32, 34, and 36. The composition will substantially or effectively seal a portion of the vaginal canal 26. In this regard, the composition includes a material selected from polyurethane, silicone, a hydrogel, temperature sensitive polymeric materials, ion sensitive materials, bioelastic polypeptide polymers, a multicomponent reverse thermo-sensitive polymeric system such as that shown and described in PCT publication WO 03/017972, the contents of which are incorporated by reference, a fibrous absorbent structure resembling an open cell polymeric foam such as that shown and described in U.S. Pat. No. 6,261,679 the contents of which are incorporated herein by reference.

The polyurethane material can be an elastomeric or foam material than can be prepared using a relatively low amount of water. A possible polyurethane material is described in U.S. Pat. No. 5,164,421, the contents of which are incorporated herein by reference.

A silicon composition can include a medical grade silicone elastomer. In this aspect, the uncured silicone elastomer in a fluid state is blended with a catalyst for solidifying the elastomer and a dilution fluid to control the viscosity during delivery and to control the modulus of elasticity of the cured solidified material. The silicone elastomer is self-curing and, upon curing has a modulus of elasticity that is substantially the same as the vagina 12 so that the insert, upon solidifying remains resilient, can act as a physical support yet not cause discomfort.

The silicone can also include an organosilicon rubber, desirably a foamable polymer where the foam includes a resilient, semi-rigid, closed-cell foam.

The composition of the present disclosure can also include a hydrogel. The term “hydrogel” as used in the specification refers to water-containing gels. These materials, when contacted with a body fluid, such as physiological fluids, swell at least 20% in volume. The amount of swelling of the hydrogel material in contact with the body fluid is desirably preferably at least 40%, more desirably at least 80% and can be in the range between 20 and 300% depending on the material used, amount of fluid to which the device is exposed and the like.

The material should be essentially inert and harmless to the body fluid and surrounding tissues and should remain intact, that is, should not be absorbed by the human or animal body into which the device is inserted. The material in the dry state should be essentially elastic and plastic only to a very small extent. In its unswollen or non-hydrated state it can be rigid, semi-rigid, or stiff, but should preferably soften upon swelling and retain a resilient property.

Suitable hydrogels are polymers and copolymers of the acrylic type such as cross-linked polyacrylamide and polymers and copolymers of acrylic and methacrylic esters having at least one hydroxy radical in the side chain. A preferred monomer is 2-hydroxy-ethyl-methacrylate; other preferred monomers are monometharylic esters of di- or triethylene glycol of 2,3-dihydroxypropane. As cross-linking agents, polyfunctional acrylates, such as the esters of the same glycols, e.g. ethylene glycol-bis-methacrylate, are useful. Materials useful in accordance with the present disclosure as well as a process for their preparation are described in the U.S. Pat. No. 3,943,045, the disclosure of which is hereby incorporated by reference.

For example, a polymer of vinyl pyrroildone and nylon in which liquid nylon or bulk nylon is polymerized with vinyl pyrrolidone, the nature of the reaction possibly being a graft polymerization or polymerization followed by crosslinking. The resulting material can be termed a hydrogel. Using such a material, the swelling and the water absorption power can be altered by changing the amounts of nylon and vinyl pyrrolidone to be polymerized. As an example, three (3) parts vinyl pyrrolidone and 1 part nylon forms a polymer having an expansion factor of 1.48, that is, it swells 48% in water, with a water content of 66%; five (5) parts vinyl pyrrolidone and 1 part nylon forms a polymer having an expansion factor of 1.78 with a water content of 78%. Other variations in monomer proportions will produce corresponding changes in the properties of the resulting hydrogel and are easily determined by one skilled in the art.

As noted above, the composition can include a temperature sensitive polymeric material. Temperature sensitive polymeric materials are described in PCT WO 98/29501, the entire contents of which are incorporated herein by reference to the extent they do not conflict herewith.

The temperature sensitive polymeric materials can be based on modified hydroxypropylcellulose compositions. In particular, it is believed that methylated hydroxypropylcellulose (m-HPC) compositions (not methylhydroxypropylcellulose (MHPC), a copolymer of methylcellulose and hydroxypropylcellulose) will be effective for use as the insert of the present disclosure. These materials are described in U.S. Pat. No. 5,770,528, the entire contents of which are incorporated herein by reference to the extent they do not conflict herewith. It is noted that these materials can also be considered an ion sensitive material.

m-HPC is produced by methylation of HPC with methylating agents such as dimethylsulfate or methylchloride. The cloud point of m-HPC can be precisely controlled by the degree of methylation. Unlike HPC that, when precipitated in water, forms a supramolecular helical structure along the backbone leading to a very loose, open precipitate with no gel-like property, m-HPC precipitates as a solid mass with a very low level of hydration. This precipitation behavior can be caused by the introduction of methyl groups, which disrupts the helical structure and increases overall hydrophobicity of the polymer chain. m-HPC has cohesive energy and gel strength comparable to poly(N-isopropylacrylamide) (IPAM), above the triggering point. As used herein the phase “triggering point” or “trigger temperature” refers to the LCST or cloud point temperature.

LCST polymers are Low Critical Solution Temperature polymers. A LCST polymer has the property of being less soluble at increased temperatures than at lower temperatures. The polymer has a lower critical solution temperature at which significant insolubilization occurs. A LCST polymer can have a single LCST point. In some circumstances, however, it is appropriate to use a polymer solution that provides two different LCST points, either as a result of including two LCST polymers having different LCST points or as a result of using a polymer having two different LCST points. It will be appreciated that when the polymer or polymer blend provides more than one LCST point there can be two different zones at which insolubilization and/or solubilization occurs or there can be a wide range of conditions at which these effects occur.

LCST polymers and their solution properties are well known and are described in, for instance, Priest et al Chapter 18 in “Reversible polymeric gels and related systems”, American Chemical Society, 1987 and in U.S. Pat. Nos. 3,244,640, 3567,650 and 3,594,326, W092/20771 and JPB-92034983 and JP-B-92034985, JP-A-04139206 and in Galaev and Mattiasson, Enzyme Microb, Technol., 1993, 15, 354-366. Suitable monomers and polymerization techniques are described in U.S. Pat. No. 5,147,923.

The LCST polymer can be a naturally occurring polymer such as certain cellulose derivatives, such as the methyl, hydroxypropyl and mixed methyl/hydroxypropyl cellulose ethers. It is generally preferred that the LCST polymer to be a synthetic polymer formed by polymerization of what can be termed an LCST monomer (or more than one such monomer), optionally as a copolymer with one or more further types of monomer. Suitable LCST monomers include N-alkylacrylamide, N,N-dialkylacrylamide, diacetone acrylamide, N-acryloylpyrrolidine, vinylacetate, certain (meth) acrylate esters (especially hydroxypropyl esters), styrene, and various other vinyl monomers, especially N-vinylimidazoline and the like.

When the LCST polymer is copolymer with other monomers, the co-monomer is usually hydrophilic and can be non-ionic or ionic. Suitable non-ionic monomers include acrylamide (ACM), substituted acrylamides, for instance those with one or two aliphatic N-substituents, some of which, such as N,N-dimethyl acrylamide (NNDMACM), can contribute to LCST properties, hydroxyethyl acrylate, vinylpyrrolidine and hydrolysed vinyl acetate. Anionic or cationic monomer can be used in place of or in addition to the non-ionic co-monomer to form a copolymer or terpolymer respectively with the one or more LCST monomers. Suitable anionic monomers include ethylenically unsaturated carboxylic or sulphonic acid monomers, for example (meth) acrylic acid and alkaline salts thereof, and 2-acrylamido methyl propane sulphonic acid. Suitable cationic monomers include dialkyl amino alkyl (meth) acrylates and acrylamides as acid addition products of quaternary ammonium salts, for example dialkylaminoethyl (meth) acrylate acid addition salts.

Diacetone acrylamide (DAAM), N,N-dimethyl acrylamide (NNDMACM), N-isopropyl acrylamide (NIPA) and N-hydroxypropyl acrylamide are particularly useful LCST monomers. LCST polymers that can be used include DAAM/ACM copolymers, NNDMACM/NIPA copolymers, NNDMACM/ACM copolymers, NIPA/ACM copolymers and poly-N-isopropylacrylamide (poly-NIPA) homopolymer.

Ion sensitive polymers are described in U.S. Pat. No. 6,602,955, the contents of which are incorporated herein by reference to the extent they do not conflict herewith.

It is noted above that bioelastic polymers can be useful as the composition of the present disclosure. Bioelastic polymers are described in U.S. Pat. No. 5,520,672, the contents of which are incorporated herein by reference. The bioelastic polymers are capable from changing from a condensed to a swollen state upon contact with a pre-selected physiological condition (such as temperature or salt content) so that the inverse temperature transition point of the bioelastomer is selectively located either above or below the ambient temperature of the local environment in which the bioelastomer is found; this allows selective unfolding and disassembly of polymer matrices to favor absorbency or brings about a contraction that causes the hydrophobic properties of the elastomers to take precedence.

The bioelastic polymers can include elastomeric units selected from the group consisting of bioelastic pentapeptides, tetrapeptides, and nonapeptides. By selecting the side changes present in the polymer portion of the composition, control is possible over the absorbence properties of the composition including the ability to the absorbent to retain absorbed liquid under stresses and external pressures. Proper selection of hydrophobic (apolar) and polar residues, which is known by one skilled in the art, will provide the resulting polymer with the property such that it is soluble in water at low temperatures but as the temperature is raised through a transitional range, they aggregate into condensed, more-ordered states.

The above materials should be selected such that the temperature at which they form into a semi-solid material should be greater than the temperature at which they do not have a semi-solid form. Desirably, the temperature at which they form into a semi-solid material is from about 35° to about 39° C., more desirably about 37° C. The temperature at which the material can be delivered to the vaginal canal is from about 16° to about 27° C., desirably about 20° C.

With respect to each of the above materials, one skilled in the art can select the appropriate material and/or modify the above material to render it more hydrophobic or more hydrophilic depending on the end use of the insert that is formed from the composition. For example, where the insert is to be used as a urinary incontinence insert, the insert is formed from a composition that is non-absorbent and desirably hydrophobic. On the other hand, where the insert is to be used as a catamenial insert, the insert is formed from a composition that is absorbent.

The disposable device 100 can also include a cover sheet 105 and a withdrawal device 110. The cover sheet 105 is configured to partially or fully envelop the composition. The cover sheet 105 can be liquid-permeable or liquid-impermeable. For example, when the cover sheet 105 is liquid-impermeable, it serves to block body fluids from contacting any materials disposed within the cover sheet 105. However, because the composition can be moisture-stable, it is not necessary in every aspect for the cover sheet 105 to be liquid-impermeable.

Depending on the materials used in the composition, a cover sheet 105 might be advisable or even necessary to ensure that the entire disposable device 100 is removed from the vagina 12 without leaving a portion of the composition behind. In addition, a cover sheet 105 can be desirable to act as a barrier between the composition and the vagina 12. Further, a cover sheet 105 can be used to limit the spread within the vagina 12 of the composition when the liquid composition is initially inserted. A cover sheet 105 can provide a smooth outer surface that can be mechanically or chemically treated to facilitate insertion and/or removal of the disposable device 100 to or from a woman's vagina 12. Finally, a cover sheet 105 can provide a material to which a string or other withdrawal device 110 can be anchored to avoid pull-out of the string or other withdrawal device 110 from the disposable device 100. In any of these aspects, the cover sheet 105 can be configured to be substantially or entirely impermeable to the composition such that the composition remains contained within the cover sheet 105 throughout insertion, use, and removal.

Suitable cover sheet materials include polyolefins such as spunbonds and bonded-carded webs, polyesters, polyethylene, polypropylene, silicon, polystyrene, polyurethane and the like. In some desirable aspects, the cover sheet 105 is a thermoplastic polymer film that is hydrophobic. One particularly suitable cover sheet material is CDR-421034 Perforated Film, a 21 gsm polyethylene blend film that is single sided vacuum aperture (available from Texol s.r.l., having a place of business located in Alanno Scalo (PE), Italy). Another particularly suitable cover sheet material is EX-1824027, a 24 gsm polyethylene blend film that is dual apertured (available from Texol s.r.l.). Yet another particularly suitable cover sheet material is EM-5218067, a dimple-embossed film that has no apertures (available from Texol s.r.l.).

The cover sheet 105 can further be formed from an apertured thermoplastic film having either a two-dimensional or a three-dimensional thickness. Apertured thermoplastic films are available from several commercial vendors including Pantex Sri, Pantex Sud srl, Via Terracini snc, having an office at 51031 Agliana, Pistoia, Italy and Applied Extrusion Technology having a mailing address of P.O. Box 582, Middleton, Del. 19709.

The cover sheet 105 can include a fibrous nonwoven material that includes a combination of synthetic fibers and natural fibers. The synthetic fibers include but are not limited to fibers such as polyester, polyolefin, nylon, polypropylene, polyethylene, polyacrylic, cellulose acetate or bicomponent fibers. Natural fibers include but are not limited to cotton and rayon. In general, the natural fibers provide ready absorption, while the synthetic fibers balance the capillary strength of the material, enabling the disposable device 100 to more readily slip against moist tissue, resulting in easier insertion and removal, hence removal comfort. The ratio of synthetic fibers to natural fibers can fall in the range of from about 90:10 to about 20:80 or even 30:70. Alternatively, the ratio of synthetic fibers to natural fibers fall in the range of from about 80:20 to about 25:75 or even from about 70:30 to about 40:60.

The synthetic fibers can have hydrophobic and hydrophilic finishes. The synthetic fibers can be inherently hydrophilic, or can be treated to provide such properties. The combination of fibers can be formulated with some level of inherently hydrophobic fiber or hydrophobic treated fiber as well, as long as it does not significantly diminish the fluid wicking strength of the cover sheet 105.

The nonwoven cover sheet 105 of the present disclosure can be mechanically altered in one or more directions in order to reduce the aggressiveness of the material. Any known means of mechanically altering films or nonwovens can be used in developing cover sheets 105 useful in the present disclosure. Mechanically altering this includes the well-known processes such as ring rolling or “pre-corrugating”, SELFing, and/or aperturing. One method of SELFing is disclosed in U.S. Pat. No. 5,518,801 to Chappell. Known methods of aperturing include hot pin (e.g., U.S. Pat. No. 5,188,625 to Van Iten et al.), slit and stretch (e.g., U.S. Pat No. 5,714,107 to Levy) and selectively aperturing (e.g., U.S. Pat. No. 5,916,661 to Benson at al.). The reduction in surface area of contact, reduced capillary strengths (due to larger pores after stretching/aperturing), and also the elastic nature of the resulting fabrics, can individually and combine to reduce the shear force from vaginal tissue, and improve removal comfort (and Agar shear values) while still maintaining good fluid handling. Mechanically altering materials that normally have very high agar shear values, such as those including a high percentage (even 100%) of rayon, enables the use of a wider variety of materials having good wicking abilities since mechanically altering can lower the agar shear value to the desirable range.

The material can be made via any number of techniques. Commonly, carded webs that are hydroentangled, thermally bonded, needled, and resin-bonded have application. The blending and layering of the synthetic and natural fibers is well known in the art. In the case of resin bonded materials, the resin bonding agent can be used in place of the synthetic fibers as the method for tempering the aggressiveness of the natural fiber matrix. In this case, all natural fiber can be used with a significant portion of synthetic binder (10-30% is common). A binder that reduces the adhesion to tissue, yet doesn't unacceptably degrade the wicking performance, would be acceptable. The binder could be of a wide variety to include, but not be limited to, acrylates, acetates, styrene-isoprene, styrene-butadiene, polyvinyl, alcohols, modified starches and the like.

Another technique to create the cover sheet 105 would utilize spunbond and meltblown processes. This technique would create a layer of less aggressive synthetic fibers that would be layered onto and intermeshed with a carded web of natural fibers.

The basis weight of the cover sheet 105 can be at least 10 grams per square meter, optionally from about 10 to about 60 grams per square meter, alternatively from about 15 to about 30 grams per square meter.

The cover sheet 105 possesses a horizontal wicking capacity (described in the test method below) greater than about 1, alternatively greater than about 3 grams of fluid per gram of cover sheet 105. Yet another aspect includes a cover sheet 105 having a horizontal wicking capacity of from about 6 to about 15 or even from about 8 to about 10 grams of fluid per gram of cover sheet 105.

In one aspect, the cover sheet 105 is 50% rayon, 50% polyester hydroentangled available as BBA 140-027. Another aspect includes a material that is dual layered with an outside and inside layer, made in accordance with U.S. Pat. No. 5,273,596. In this case, the outside layer is a 75% hydrophilically-treated polypropylene with a 2.2 dpf and 25% 1.5 dpf rayon. The inside layer is 25% hydrophilically-treated polypropylene with a 2.2 dpf and 75% 1.5 dpf rayon. The basis weights of the layers can vary, having from about 10 to about 15 grams per square meter in each layer. The resultant material is a 50% rayon 50% polypropylene thermally bonded blend with a basis weight from about 20 to about 30 grams per square meter. Both materials are produced by BBA Corporation of South Carolina, U.S.A.

In other aspects of the present disclosure, the cover sheet 105 can be made from foams, woven materials, any other suitable cover material, or any combine on of the cover materials described herein. For example, the cover sheet 105 can include a foam material such as those obtainable from The Dow Chemical Company of Midland, Mich. U.S.A. Representative foam materials are described in U.S. Pat. Nos. 6,627,670 82 to Mork et al., 6,071,580 to Bland et al., 7,439,276 82 to Strandburg et al., and in PCT Publication Nos. WO2008/036942A2 to Vansumeren et al., WO2007/011728A2 to Kim at al,, WO2008/052122A1 to Manning, and WO2008/100842A1 to Stockton et al., which are incorporated herein in their entirety by reference thereto to the extent they do not conflict herewith.

Suitable foam materials can also include various types of foams, including, but not limited to thermoplastic foams, high internal phase emulsion (HIPE) foams and inverse high internal phase emulsion (I-HIPE) foams, and other suitable polymeric foam including, but not limited to, those disclosed in U.S. Pat. Nos. 7,053,131 to Ko et al., 7,358,282 to Krueger et al., and 5,692,939 to DesMarais et al., and in U.S. Patent Application Publication No. US2006/0148917 to Radwanski et al., which are incorporated herein in their entirety by reference thereto to the extent they do not conflict herewith. Further examples of suitable absorbent foam materials are described in U.S. Patent Application Publication No. US200610246272 to Zhang et al., which is incorporated herein in its entirety by reference thereto to the extent it does not conflict herewith.

The cover sheet 105 can be formed from woven or nonwoven material having a porous substrate. Woven material includes textile fabrics that can be made from rayon, cotton, polyolefins or ether synthetic yarns. The synthetics can be either staple or continuous filaments.

In some aspects, the surface of the cover sheet 105 can include apertures and/or can be embossed with grooves, dimples, dots, and the like that can reduce the surface contact area when inserted into the vagina. One advantage of the disposable device 100 of the present disclosure is that the cover sheet 105 has a uniquely low surface contact area that can reduce or eliminate irritation to the vaginal tissues, can reduce or eliminate the occurrence of the disposable device 100 becoming un-removable by the user, and can provide a disposable device 100 that is easier for women to insert into and remove from their bodies, without the need for surface treatments such as mineral oil.

The cover sheet 105 can be folded over upon itself and be bonded to itself to form a longitudinal bond. Suitable bonding means include heat, pressure, ultrasonic, adhesive, and the like. In some aspects, the cover sheet 105 can be simply folded over upon itself. In other aspects of the present disclosure, the cover sheet 105 can be sealed at the ends to form end seals using any suitable bonding means including heat, pressure, ultrasonic, stitching, adhesives, and the like.

The cover sheet 105 substantially covers the surface of the disposable device 100. “Substantially covers” in this case means that the cover sheet 105 covers at least about 75%, optionally at least about 90% of the surface area of the surface. The cover sheet 105 can be wrapped around the longitudinal axis or the transverse axis. In other aspects, two separate pieces of cover sheet 105 can sandwich the disposable device 100.

The cover sheet 105 can be joined to the disposable device 100 by any variety of means. The cover sheet 105 can be joined to itself or to the disposable device 100. For example, one portion of the cover sheet 105 can be joined to an opposed portion of the cover sheet 105 or the disposable device 100 using any suitable adhesive or heat/pressure bonding means. Such adhesive can extend continuously along the length of attachment or it can be applied in a “dotted” fashion at discrete intervals. One method of heat bonding includes thermally bonding, fusion bonding, or any other suitable means known in the art for joining such materials. Alternatively, the cover sheet 105 can be joined to the disposable device 100 by stitching. Such stitching can use cotton or rayon thread.

In one aspect, the disposable device 100 of the present disclosure can include a withdrawal device 110. The withdrawal device 110 can be joined to the disposable device 100 for removal of the disposable device 100 after use. The withdrawal device 110 can be joined to at least the disposable device 100 and extends beyond at least the withdrawal end. Any of the withdrawal devices 110 currently known in the art can be used as a suitable withdrawal mechanism. In addition, the withdrawal device 110 can take on other forms such as a ribbon, loop, tab, or the like. The withdrawal device 110 can be integral with the disposable device 100.

It is also contemplated that the withdrawal device 110 can be provided with the composition such that upon delivery of the composition to the vaginal canal 26, the withdrawal device 110 can also be delivered to the vaginal canal 26 so that as the composition solidifies, the withdrawal device 110 is secured within the insert formed as a result of the solidification of the composition.

The withdrawal device 110 can be non-absorbent along at least the location of attachment to the disposable device 100. As used herein, the term “non-absorbent” refers to a structure that does not retain a significant portion of deposited fluid in its structure. The entire withdrawal device 110 or other withdrawal mechanism can be made non-absorbent, if desired. The materials including the withdrawal device 110 can be inherently non-wettable or hydrophobic, or they can be treated to provide such properties. For example, a coating of wax can be applied to the withdrawal device 110 to decrease or eliminate its absorbency. The withdrawal device 110 need not necessarily be non-wicking, even if a non-absorbent withdrawal device 110 is desired. For example, it can be desirable to provide a withdrawal device 110 in which at least a portion of the withdrawal device 110 has a tendency to wick deposited fluid upwardly toward the withdrawal end of the disposable device 100 and into the structure thereof.

The withdrawal device 110 can be attached in any suitable manner known in the art including sewing, adhesive attachment, or a combination of known bonding methods. The withdrawal device 110 can be joined to any suitable location on the disposable device 100.

The disposable device 100 of the present disclosure can be inserted digitally or through the use of an applicator. Any of the currently available tampon or urinary incontinence device applicators can also be used for insertion of the disposable device 100 of the present disclosure. Such applicators are typically a “tube and plunger” type arrangement and can be plastic, paper, or other suitable material. Additionally, a compact-type applicator is also suitable.

The composition including one of more of the materials described above, along with the cover sheet 105, is contained in a delivery device that will contain the composition until delivery to the vaginal canal. The delivery device can include, but is not limited to an aerosol, squeeze tube, pump, or other applicators (known in the art). Additionally a suppository, capsule, effervescent or foam producing tablet or other suitable device to deliver the composition into the vaginal canal 26 where upon introduction, the composition will solidify to a semi-solid or solid form having elasticity and resiliency.

The present disclosure therefore contemplates a method of providing a vaginal insert that includes providing a delivery device that contains a composition and dispensing the composition to the vaginal canal 26 where the composition is fluid like when outside the vagina 12 but that upon contact with the interior surface of the vaginal canal 26 and rugae will solidify to form a malleable, resilient insert suitable for use as an incontinence insert or catamenial insert depending upon the properties of the composition and independent of the location of the insert.

While the disclosure has been described in conjunction with specific aspects it is to be understood that many alternatives, modifications, and variations will be apparent to those skilled in the art in light of the foregoing detailed description. It is therefore intended that the foregoing description be regarded as illustrative rather than limiting, and that it be understood that it is the following claims, including all equivalents, that are intended to define the spirit and scope of this disclosure.

What is claimed:

1. An anatomically-conforming vaginal insert formed from a composition wherein the composition includes a material that is introduced into a vaginal canal as a viscous material a first temperature and after insertion into the vaginal canal becomes a semi-solid at a second temperature greater than the first temperature, wherein the first temperature is about 20° C. and the material has increased solidity at the second temperature as compared to the first temperature, and wherein the composition is disposed within a cover sheet.

2. The vaginal insert of claim 1, wherein the cover sheet is made from at least one of a nonwoven material, a film material, a foam material, and a woven material.

3. The vaginal insert of claim 1, wherein the vaginal insert is formulated with a withdrawal device.

4. The vaginal insert of claim 3, wherein the withdrawal device is attached to the cover sheet.

5. The vaginal insert of claim 1, wherein tine composition is selected from the group consisting of a material selected from polyurethane, silicone, a hydrogel, temperature sensitive polymeric materials, ion sensitive materials, bioelastic polypeptide polymers, a multi-component reverse thermo-sensitive polymeric system, and a fibrous absorbent structure resembling an open cell polymeric foam.

6. The vaginal insert of claim 5, wherein the material is a temperature sensitive polymeric material having a low critical solution temperature such that the second temperature is about 37° C.

7. The vaginal insert of claim 1, therein the insert is hydrophobic.

8. The vaginal insert claim 7, wherein the vaginal insert is an incontinence insert.

9. The vaginal insert of claim 8, wherein the vaginal insert supports musculature around a bladder neck.

10. The vaginal insert of claim 1, wherein the composition expand upon insertion into the vaginal canal.

11. The vaginal insert of claim 1, wherein the material is a polymer having lower solubility at the second temperature than the first temperature.

12. The vaginal insert of claim 1, wherein the material is a liquid, semi-soft liquid, gel, paste, foam or other viscous material at a temperature of about 16° to about 27° C., but forms a semi-solid material at a temperature of about 35° to about 39° C.

13. The vaginal insert of claim 1, wherein the cover sheet is liquid-permeable.

14. The vaginal insert of claim 1, wherein the cover sheet is composition-impermeable.

15. The vaginal insert of claim 1, wherein the material is selected from the group consisting of a material selected from polyurethane, silicone, temperature sensitive polymeric materials, ion sensitive polymeric materials, and humidity activated materials having a shape memory.

16. An anatomically-conforming vaginal insert formed from a composition that is introduced into a vaginal canal as a liquid, semi-soft liquid, gel, paste, foam or viscous composition at a temperature of about 16° to about 27° C., and after insertion into the vaginal canal becomes a solid or semi-solid material at a temperature of about 35° to about 39° C. wherein the composition is disposed within a cover sheet, and wherein the vaginal insert is formulated with a withdrawal device.

17. The vaginal insert of claim 16, wherein the cover sheet is made from at least oneof a nonwoven material, a film material, a foam material, and a woven material.

18. The vaginal insert of claim 16, wherein the withdrawal device is attached to the cover sheet.

19. The vaginal insert of claim 16, wherein the cover sheet is liquid-permeable.

20. The vaginal insert of claim 16, wherein the cover sheet is composition-impermeable.
